(19) United States
(12) Patent Application Publication
(10) Pub. No.: US 2007/0111959 A1
(43) Pub. Date: May 17, 2007

(54) Combination gene for use in inhibiting cancerous cell growth

(76) Inventors: James Yockman, West Valley City, UT (US); Sung Wan Kim, Salt Lake City, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/281,722

(22) Filed: Nov. 17, 2005

(51) Int. Cl. A61K 48/00; C12N 15/09

(52) U.S. Cl. 514/44; 435/455

(57) ABSTRACT

An expression vector for use in inhibiting cancerous cell growth includes the following: (a) a first promoter nucleotide sequence; (b) a first coding nucleotide sequence encoding for a receptor that interacts with an angiogenic growth factor operatively linked to the first promoter sequence; (c) a second promoter nucleotide sequence upstream or downstream from the first promoter nucleotide sequence; and (d) a second coding nucleotide sequence encoding for a cytokine operatively linked to the second promoter nucleotide sequence. The expression vector can be used in a method of inhibiting growth of cancerous cells by the following: (a) providing a dual expression vector; (b) introducing the expression vector into at least one cell capable of expressing at least one of the receptor or cytokine; and (c) producing the receptor and cytokine.

### BACKGROUND OF THE INVENTION

1. The Field of the Invention

The present invention is related to genes for use in inhibiting the growth of cancerous cells. More particularly, the present invention is related to the inclusion of a combination of nucleotide coding sequences on a single expression vector that are each driven by a separate promoter. The nucleotide coding sequences can encode for soluble receptors, such as VEGF-2R, which interact with angiogenic factors, and encode for cytokines, such as IL-2, that are involved in immunological responses against cancerous cells.

2. The Related Technology

Renal cell carcinoma continues to be to be a major health issue, and predictions indicate the disease will become increasingly prevalent over time. Renal cell carcinoma is considered to be a leading cause of cancer-related deaths where survival rates drastically decrease as the disease progresses. For example, the 5-year survival rates initially reported by Robson et al. in 1969 were as follows: 66% for stage I; 64% for stage II, but contained within Gerota fascia; 42% for stage III; and only 11% for stage IV, which is considered to be a late detection (Robson C J, Churchill B M, Anderson W: The results of radical nephrectomy for renal cell carcinoma. J Urol 1969 March; 101(3): 297-301). These survival statistics have remained essentially unchanged for several decades, except for stage I having a modest increase in survival during early detection.

Additionally, renal cell carcinoma is characterized by a lack of early warning signs, diverse clinical manifestations, and possible resistance to radiation and chemotherapy, all of which illustrate the complexities faced in providing suitable treatments. Exemplary treatment options include surgery, radiation therapy, chemotherapy, hormonal therapy, immunotherapy, or combinations of these treatments. However, no hormonal or chemotherapy has been established as a standard of care, which has left viable treatments to surgery and immunotherapy.

Accordingly, radical nephrectomy of localized renal cell carcinoma involves the complete resection of everything within the Gerota fascia, which includes the kidney itself, perirenal fat, and ipsilateral adrenal gland. As such, severe long-term complications can arise from these surgical procedures. On the other hand, immunotherapy with cytokines, such as interferons and interleukins, has been explored. Interferons, such as interferon-alpha, have exhibited a direct anti-proliferative effect on renal cell carcinoma in vitro, but have been minimally successful as therapies. Additionally, interleukins, such as interleukin-2 (“IL-2”), decreases renal cell carcinoma by activating lymphoid cells rather than directly inhibiting tumor proliferation. IL-2 has remained the gold standard for renal cell carcinoma treatment even though there is a significant chance of systemic toxicity when administered at high concentrations.

Recently, advances in biotechnology have led to the development of potential gene therapies for various ailments that utilize genes that encode for therapeutic proteins such as interleukins, interferons, and anti-antiogenic agents. The use of genes as therapeutic agents has the potential of providing the benefit of the encoded protein without the toxicities and immunological responses associated with protein-based immunotherapies. However, genes configured to be suitable for delivery to a patient and capable of expressing proteins at therapeutic levels continue to be researched and developed.

Therefore, it would be advantageous to have a combination therapy that utilizes genes encoding for cytokines and anti-angiogenic agents.

### BRIEF SUMMARY OF THE INVENTION

Generally, embodiments of the present invention can include expression vectors that can be used in methods of inhibiting the growth of cancerous cells. The expression vectors can be characterized by having different coding nucleotide sequences that are driven by separate promoters. Also, the coding nucleotide sequences encode for different types of polypeptides that can be used as anti-cancer agents.

In one embodiment, the present invention includes an expression vector for use in inhibiting cancerous cell growth, and includes at least two coding nucleic acid sequences. Accordingly, the vector can include the following: (a) a first promoter nucleotide sequence; (b) a first coding nucleotide sequence operatively linked to the first promoter sequence, wherein the first coding nucleotide sequence encodes for a receptor that interacts with an angiogenic growth factor; (c) a second promoter nucleotide sequence upstream or downstream from the first promoter nucleotide sequence; and (d) a second coding nucleotide sequence operatively linked to the second promoter nucleotide sequence, wherein the second coding nucleotide sequence encodes for an immunomodulatory cytokine. The expression vector can be particularly useful in inhibiting the growth of cancerous renal cells when the receptor binds with the angiogenic growth factor so as to reduce angiogenesis associated with renal tumor growth, and/or when the cytokine activates a physiological response against the cancerous cells.

In one embodiment, the present invention includes an expression vector for use in inhibiting cancerous cell growth. As such the vector can include the following: (a) a first promoter nucleotide sequence operatively linked to a first coding nucleotide sequence encoding for a first polypeptide that interacts with an angiogenic growth factor so as to inhibit angiogenesis; and (b) a second promoter nucleotide sequence operatively linked to a second coding nucleotide sequence encoding for a second polypeptide that activates a physiological response against the cancerous cells. Also, the second promoter sequence can be located upstream or downstream from the first promoter sequence. The first polypeptide and second polypeptide can be independently expressed by the second coding nucleotide sequence not being operatively linked to the first promoter sequence and the first coding sequence not being operatively linked to the second promoter nucleotide sequence.

In one embodiment, the present invention includes a method of inhibiting the growth of cancerous cells by use of an expression vector. The method can be employed by providing an expression vector that includes the at least two coding nucleotide sequences each operatively linked to a separate promoter, wherein the at least two coding nucleotide sequences encode for an anti-angiogenic receptor or a cytokine. The expression vector can be introduced into at least one cell capable of expressing at least one of the receptor or cytokine. The growth of the cancerous cells can be inhibited by producing the receptor and/or cytokine encoded on the expression vector. Additionally, the combination of the receptor and cytokine can inhibit angiogenesis and induce a physiological anti-cancer response so as to be more efficacious than the expression of either alone.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a vector map that illustrates an embodiment of an expression vector having a coding nucleotide sequence for a soluble receptor for an angiogenic factor and a coding nucleotide sequence for a cytokine;

FIG. 2 is a vector map that illustrates an embodiment of an expression vector having a coding nucleotide sequence for a soluble receptor for an angiogenic factor and a coding nucleotide sequence for a cytokine;

FIG. 3 is a flow diagram illustrating an embodiment of a method for inhibiting cancerous cell growth;

FIG. 4 is a flow diagram illustrating an embodiment of a method for method for inhibiting cancerous cell growth;

FIG. 5 is a flow diagram illustrating an embodiment of a method for inhibiting cancerous cell growth;

FIG. 6 is a bar graph illustrating IL-2 expression resulting from an embodiment of in vitro transfection;

FIG. 7 is a graph illustrating RENCA tumor volume resulting from an embodiment of local in vivo gene delivery and transfection;

FIG. 8 is a graph illustrating the survival of mice bearing renal cell tumors n embodiment of systemic in vivo gene delivery and transfection; and

FIG. 9 is a bar graph illustrating the number of lung metastases forming n embodiment of systemic in vivo gene delivery and transfection.

### DETAILED DESCRIPTION OF THE PREFERRED EMBODIMENTS

Generally, embodiments of the present invention relate to expression vectors that have multiple coding nucleotide sequences for different polypeptide products. Also, each coding nucleotide sequence is operatively linked to a separate promoter nucleotide sequence that is not operatively linked to any other coding nucleotide sequence. Exemplary coding nucleotide sequences and promoter nucleotide sequences are provided below in the Sequence Listing, which encode for polypeptide products that interact with angiogenic factors and/or polypeptide products that induce anti-cancer cytokine-based physiological responses.

I. Polypeptides for Inhibiting Cancerous Cell Growth

The potential for genes to be used in therapies has been studied extensively in recent years. The use of a gene to provide a therapeutic benefit relies on the ability to express the protein encoded on the gene, which itself is dependent on the expression vector and the gene carrier. As such, various expression vector constructs have been shown to successfully induce protein expression in cells. This can include expressing proteins in cells that do not naturally produce such proteins, and/or inducing cells to up-regulate protein expression. Accordingly, it has been found that the delivery of useful genes can be beneficial in treating or inhibiting the growth of cancerous cells. However, the induced expression of a single polypeptide has yet to provide a therapeutic benefit in fighting cancer. Thus, an embodiment of the present invention provides an expression vector encoding for a combination of beneficial polypeptides that can be useful in treating or inhibiting the growth of cancerous cells, especially cancerous renal cells.

Cancerous cells need oxygen and nutrients, which are usually supplied by blood, in order to grow larger in size and number. As such, the proliferation of cancer cells and tumor growth needs a blood source, which can be obtained by passive diffusion when few cancer cells are present. Once a tumor or group of cancer cells reaches about 1 mm to about 2 mm in any dimension, it must develop a dedicated blood supply in order to grow larger because diffusion is no longer adequate to supply the cells with oxygen and nutrients as well as to also to take away waste materials.

Accordingly, cancer cells secrete substances that promote the formation of new blood vessels during angiogenesis, wherein the new blood vessels usually form a conduit for blood to be transferred to and from the tumor. As such, many angiogenic factors have been identified to promote angiogenesis such as, for example and not limitation, angiopoietin-1, fibroblast growth factors (“FGF”), vascular endothelial growth factors (“VEGF”), epidermal growth factors (“EGF”), platelet-derived growth factors (“PDGF”), transforming growth factors alpha and beta (“TGF-alpha” and “TGF-beta”), angiogenin, angiotropin, nitric oxide, and the like. Also, a VEGF isoform has been implicated in generating lymphatic vessels for aiding in cancer cell proliferation and/or migration.

Additionally, a number of natural angiogenesis inhibitors have been discovered, and have been used to inhibit cancer cell proliferation. Angiostatin and endostatin are polypeptides that interfere with angiogenesis. Receptors for angiogenic factors have been shown to reduce the concentration of the angiogenic factors and hence produce an anti-angiogenic effect. Examples of such angiogenic factor receptors can include vascular endothelial growth factor receptor-1 (“VEGF-R1” or “Flt-1”), vascular endothelial growth factor receptor-2 (“VEGF-R2,” “Flk-1,” or “KDR”), and vascular endothelial growth factor receptor-3 (“VEGF-R3” or “Flt-4”). Also, soluble forms of receptors for VEGF bind therewith and provide an anti-angiogenic effect. A soluble fetal liver kinase (“sFlk-1”) receptor is an example of such an anti-angiogenic receptor; however, other soluble receptors for angiogenic factors are contemplated to also be effective for inhibiting angiogenesis.

Immunophysiology provides natural mechanisms that continually search and destroy cancerous cells. As such, the immune system includes a complex chemical signaling system that induces the immunological response against tumors. One aspect of the immunological response against tumors includes the production of polypeptides in the form of cytokines, which are the chemical signalers.

Cytokines are a complex group of soluble intercellular regulatory proteins that control many aspects of the immune system, and are produced by many different types of cells. For example, cytokines activate and deactivate phagocytes and immune defense cells, and promote or inhibit a variety of innate and/or adaptive immunological responses. They are produced by virtually all cells involved in innate and adaptive immunity, but especially by T helper (Th) lymphocytes. The activation of cytokine-producing cells triggers them to synthesize and/or secrete their cytokines. The cytokines, in turn, are then able to bind to specific cytokine receptors on other cells of the immune system and influence their immunological activity in some manner.

A particular cytokine can signal a number of different types of cells and regulate a number of different functions, and multiple cytokines can carry out the same function. Cytokines that regulate innate immunity are produced primarily by mononuclear phagocytes, such as macrophages and dendritic cells, although they can also be produced by T-lymphocytes, N K cells, and other cells. The cytokines that regulate innate immunity can include, for example and not by way of limitation, tumor necrosis factor (“TNF”), interleukin-1 (“IL-1”), interleukin-6 (“IL-6”), interleukin-10 (“IL-10”), interleukin-12 (“IL-12”), interleukin-15 (“IL-15”), interleukin-18 (“IL-18”), chemokines, and Type I interferons. Cytokines that regulate adaptive immunity are produced primarily by T-lymphocytes and NK cells, and can include, for example and not by way of limitation, interleukin-2 (“IL-2”), interleukin-4 (“IL-4”), interleukin-5 (“IL-5”), interleukin-13 (“L-13”), interferon-gamma (“INF-gamma”), transforming growth factor beta (“TGF-beta”), and lymphotoxin (“LT”). Also, hematopoietic cytokines that stimulate the growth and differentiation of immature leukocytes are usually produced by bone marrow stromal cells, and include, for example and not by way of limitation, interferon-3 (“IL-3”), interferon-7 (“IL-7”), colony stimulating factors (“CSF”), and stem cell factor (“SCF”).

It has been found that various cytokines that regulate the innate and adaptive immunity as well as hematopoietic cytokines can be involved in anticancer immunological responses. While IL-2 is the most studied cytokine that demonstrates anticancer properties, it is suspected that most, if not all, of the other cytokines are, at least indirectly, involved in anticancer immunological responses. As such, while only IL-2 is exemplified herein, it is contemplated that any other cytokine can be used in the present invention.

Previously, various polypeptides with anticancer characteristics have been administered to patients with cancer in an attempt to inhibit the growth of cancer cells. Additionally, instead of administering the polypeptide itself, it has been shown that the delivery of genes that encode for the polypeptides can result in the beneficial expression of such polypeptides. Accordingly, embodiments of the present invention are directed to expression vectors that encode for a combination of polypeptides that inhibit the growth of cancer cells. More specifically, the expression vectors encode for a receptor that interacts with an angiogenic factor and a cytokine that is involved with a physiological response against cancerous cells. Thus, coding nucleotide sequences for any of the foregoing receptors and cytokines can be included in a combination expression vector in accordance with the present invention.

II. Expression Vectors

In one embodiment, the present invention includes an expression vector that encodes for the production of a combination of polypeptides that together can inhibit the growth of cancerous cells. Also, in some cases the inhibition is greater with the expression of the combination of polypeptides from a single vector compared to either polypeptide expressed alone and/or expressed from separate vectors.

Expression vectors that encode for beneficial polypeptides can be obtained or made using genetic engineering techniques well known in the art. As such, various expression vectors that encode for only one polypeptide can be genetically engineered by techniques well known in the art to include at least two polypeptides that can be beneficial in inhibiting cancerous cell growth. Accordingly, these expression vectors, as well as other expression vectors that encode for the same or different polypeptides, can be used in constructing a combination expression vector.

The expression vector pCMV-sFlt-1, which is a well-known expression vector, is an embodiment of an expression vector that includes a coding nucleotide sequence for a single beneficial polypeptide in the form of a receptor that interacts with an angiogenic factor. In this embodiment, the coding nucleotide sequence encodes for a soluble form of the Flt-1 or VEGF-R1 anti-angiogenic receptor in a human isoform. Also, the expression vector includes a CMV enhancer/promoter that drives the expression of the beneficial polypeptide. The promoter nucleotide sequence for the CMV promoter is in the Sequence Listing at SEQ ID NO: 8. The expression vector also includes other optional features such as a chimeric intron, T7 promoter, polyA signal, phage F1 region, a beta-lactamase marker gene, and various restriction enzyme sites. Accordingly, pCMV-sFlt-1 can be genetically engineered by well-known techniques to excise the promoter nucleotide sequence (e.g., CMV) and/or polypeptide coding nucleotide sequence (e.g., Flt-1) for combination into another vector. Alternatively, pCMV-sFlt-1 can be genetically engineered to have an additional promoter nucleotide sequence and coding nucleotide sequence added thereto. While a human isoform is depicted, any other VEGF-receptor from any animal source, such as mice, may be used and spliced into a combination vector. Moreover, the sFlt-1 coding sequence, which can optionally also include the CMV promoter, can be snipped from the expression vector and ligated into a combination expression vector in accordance with the present invention.

The expression vector pCMV-sFlk-1, which is a well-known expression vector, is an embodiment of an expression vector that includes a coding nucleotide sequence for a single beneficial polypeptide in the form of a receptor that interacts with an angiogenic factor. The receptor is a soluble form of the Flk-1 or VEGF-R2 murine isoform. The coding nucleotide sequence for murine soluble Flk-1 is in the Sequence Listing at SEQ ID NO: 4 and the polypeptide sequence is at SEQ ID NO: 5. Also, the expression vector includes a CMV enhancer/promoter that drives the expression of the beneficial polypeptide, wherein the priomoter nucleotide sequence for the CMV promoter is in the Sequence Listing at SEQ ID NO: 8. The expression vector can also include other optional features such as a chimeric intron, T7 promoter, polyA signal, phage F1 region, a beta-lactamase marker gene, and various restriction enzyme sites. Accordingly, pCMV-sFlk-1 can be genetically engineered by well-known techniques to excise the promoter nucleotide sequence (e.g., CMV) and/or polypeptide coding nucleotide sequence (e.g., Flk-1) for combination into another vector. Alternatively, pCMV-sFlk-1 can be genetically engineered to have an additional promoter nucleotide sequence and/or polypeptide coding nucleotide sequence added thereto.

The expression vector pCMV-IL-2, which is a well-known expression vector, is an embodiment of an expression vector that includes a coding nucleotide sequence for a single beneficial polypeptide in the form of the IL-2 cytokine, which is involved in an immunological response against cancerous cells. In this embodiment, the IL-2 cytokine is a human isoform, wherein the coding nucleotide sequence is in the Sequence Listing at SEQ ID NO: 6 and the cytokine polypeptide sequence is at SEQ ID NO: 7. Also, the expression vector includes a CMV enhancer/promoter that drives the expression of the beneficial polypeptide. The CMV nucleotide sequence is SEQ ID NO: 8. The expression vector can also include other optional features such as a chimeric intron, T7 promoter, polyA signal, phage F1 region, a lacZ alpha marker gene, ampicillin, and various restriction enzyme sites. Accordingly, pCMV-IL-2 can be genetically engineered by well-known techniques to excise the promoter nucleotide sequence (e.g., CMV) and/or polypeptide coding nucleotide sequence (e.g., IL-2) for combination into another vector. Alternatively, pCMV-IL-2 can be genetically engineered to have an additional promoter nucleotide sequence and/or polypeptide coding nucleotide sequence added thereto. While a human isoform is depicted, any other cytokine that is involved in an anticancer physiological response and from any source, such as mice, may be used and spliced into a combination vector. Moreover, the IL-2 coding sequence, which can optionally also include the CMV promoter, and/or any of the optional features can be snipped from the expression vector and ligated into a combination expression vector.

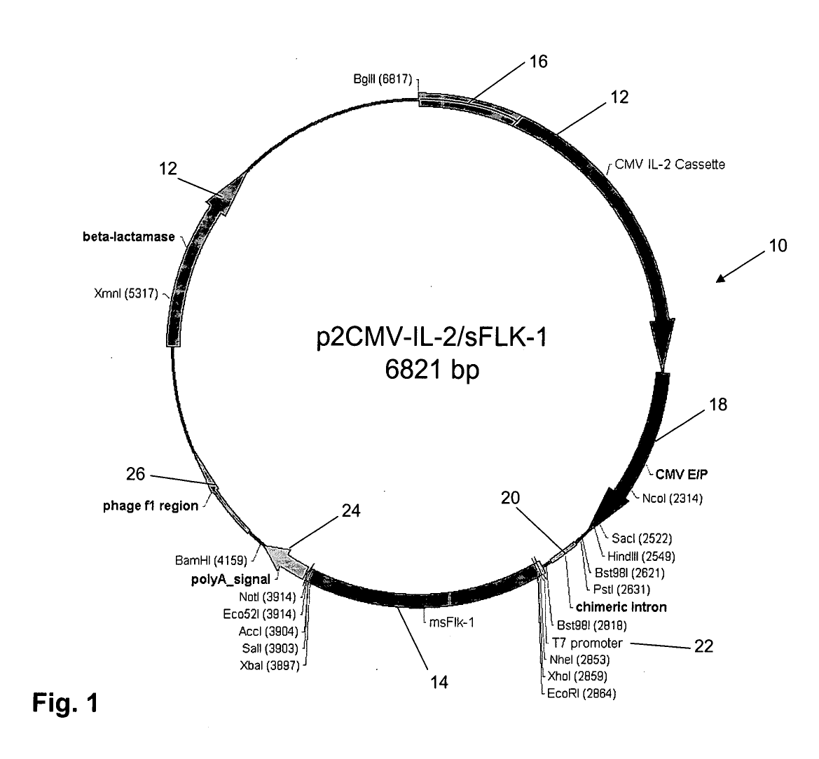

FIG. 1 illustrates an embodiment of a combination expression vector p2CMV-IL-2/sFlk-1 10 that includes a coding nucleotide sequence 12 encoding for a cytokine that is involved in an immunological response against cancerous cells and a downstream coding nucleotide sequence 14 for a beneficial polypeptide in the form of a receptor that interacts with an angiogenic factor. In this embodiment, the cytokine is a human isoform of IL-2, and the receptor is a soluble form of the murine isoform of VEGF-R2 or msFlk-1. The expression vector can include a separate CMV enhancer/promoter 16 that drives the expression of the cytokine, and a separate CMV enhancer/promoter 18 that drives the expression of the receptor. The expression vector can also include other optional features such as a chimeric intron 20, T7 promoter 22, polyA signal 24, phage F1 region 26, a lacZ alpha marker gene 28, ampicillin gene (not shown), various restriction enzyme sites (not numbered), and the like. Also, some of these features are also included in the CMV IL-2 cassette 12, 16.

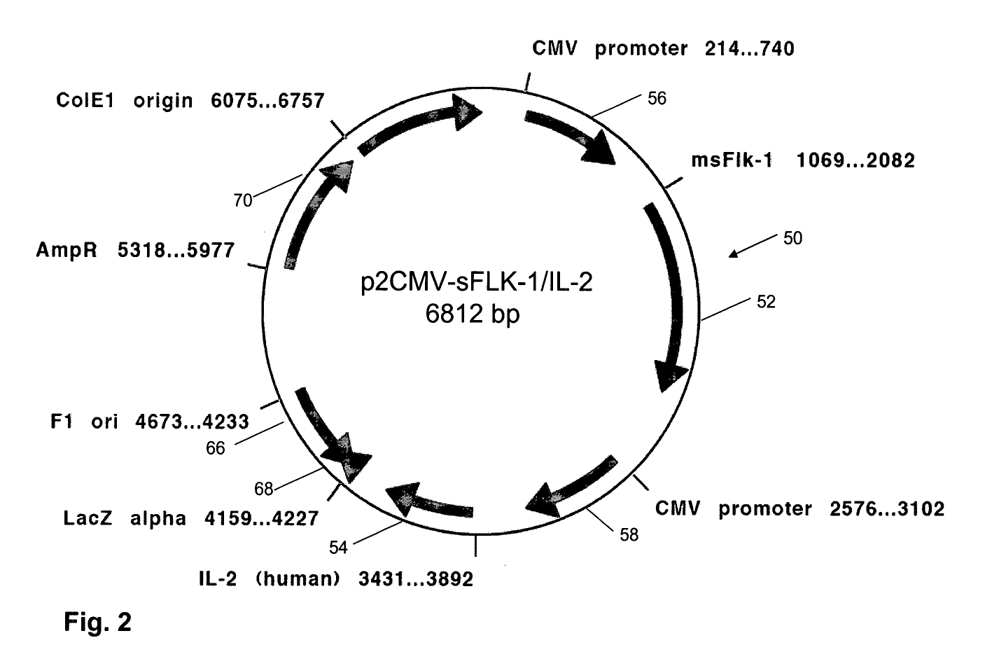

FIG. 2 illustrates an embodiment of a combination expression vector p2CMV-sFLK-1/IL-2 50 that includes a coding nucleotide sequence 52 for a beneficial polypeptide in the form of a receptor that interacts with an angiogenic factor and a downstream coding nucleotide sequence 54 for a cytokine that is involved in an immunological response against cancerous cells. Again, the cytokine is a human isoform of IL-2, and the receptor is a soluble form of the murine isoform of VEGF-R2 or msFlk-1; however, any isoform from any animal for either the cytokine or receptor can be used in a combination vector. Also, the expression vector includes a separate CMV enhancer/promoter 56 that drives the expression of the cytokine, and a separate CMV enhancer/promoter 58 that drives the expression of the receptor.

Moreover, FIG. 2 illustrates the vector map of the nucleotide sequence of SEQ ID No: 1. Additionally, the expression vector can also include other optional features such as a chimeric intron (not shown), T7 promoter (not shown), polyA signal (not shown), phage F1 region 66, a lacZ alpha marker gene 68, ampicillin gene 70, various restriction enzyme sites (not numbered), and the like.

While different embodiments of combination expression vectors in accordance with the present invention have been illustrated and described, additional embodiments that encode for other angiogenic factor receptors and/or other cytokines are within the scope of the present invention. For example, coding nucleotide sequences that encode for any of the isoforms of receptors for VEGF, such as VEGF-R1, VEFG-R2, VEGF-R3, or any other VEGF-receptors known or later developed, can be included in the combination expression vector to stimulate production of such receptors, especially in soluble form. Additionally, any coding nucleotide sequence that encodes for a receptor for any of the other angiogenic factors, such as receptors that interact with angiopoietin-1, FGF, VEGF, EGF, PDGF, TGF-alpha, TGF-beta, angiogenin, angiotropin, or nitric oxide, can be used in a combination vector to inhibit angiogenesis. Moreover, additional coding nucleotide sequences that encode for other beneficial polypeptides other than IL-2 that have anticancer activity can be included in the expression vector. Furthermore, any coding nucleotide sequence that encodes for a cytokine involved in an anticancer immunological response can be included in the expression vector.

III. Expression Vector Delivery

In order to express the proteins encoded on an expression vector, such as a combination expression vector in accordance with the present invention, the nucleic acid has to be delivered into a cell. Accordingly, various gene carriers have been constructed and implemented for such purposes. Methods of delivering genes into a cell include, chemical precipitation, ballistic penetration, micro-injection, viral carriers, and non-viral carriers such as polymers, liposomes, or lipopolymers, as well as other known processes and those yet to be discovered. While any of these delivery processes can be used for delivering expression vectors into cells in order for the encoded polypeptides to be expressed, the preferred gene carrier includes non-viral carriers. Thus, any now-known or future-developed process for effecting gene delivery for functional polypeptide expression is contemplated to be useful in embodiments of the present invention, wherein such processes of gene delivery are intended to be included herein.

In one embodiment, the present invention includes a water soluble lipopolymer (“WSLP”) as a gene carrier for the expression vector. The gene carrier is used in an amount capable of combining and packaging a nucleic acid for delivery into a cell. For example, the WSLP amine (which includes nitrogen “N”) to nucleic acid phosphate (which includes phosphorous “P”) ratio can range from I to about 35 (N:P), OZ u a° more preferably from about 10 N:P to about 30 N:P, and most preferably from about 15 N:P to about 25 N:P, wherein a N:P of about 20 is most preferred. Also, the gene carrier can be used in an amount that is effective in condensing the nucleic acid as well as effective in delivering the expression vector into a cell so that the polypeptides encoded thereon can be expressed.

IV. Cancerous Cell Growth Inhibition

Embodiments of the present invention include the use of a combination expression vector in a method of inhibiting cancerous cell growth. As such, the combination expression vector can be delivered to any type of cell in a patient that has cancer. Also, the combination expression vector can produce at least one, but preferably at least two, of the polypeptides encoded on the vector. Additionally, the combination expression vector can be delivered to cancerous cells as well as other types of cells so long as the local and/or systemic concentration of the beneficial polypeptides is increased in an amount to provide an anticancer benefit.

As used herein, the terms “inhibit,” “inhibition,” or “inhibiting” are meant to refer to the process of reducing or limiting from a full potential, especially in relation to a physiological process such as cancer cell growth. This includes a minimal reduction through fully stopping the process. For example, inhibiting the growth of cancerous cells can include reducing the amount a cell grows through fully stopping the cell growth, or possibly even killing the cell so that it no longer is able to grow. Additionally, inhibiting the growth of cancerous cells can include reducing the amount of cell division so that the normally geometric growth of a tumor by cell division is reduced through fully stopped. That is, this allows for some cancerous cells to continue to divide and propagate, but it also reduces the amount of division and propagation from the full geometric potential. Moreover, the inhibition of cancerous cell growth can include reducing the number of cells so that a tumor actually regresses and even is completely killed.

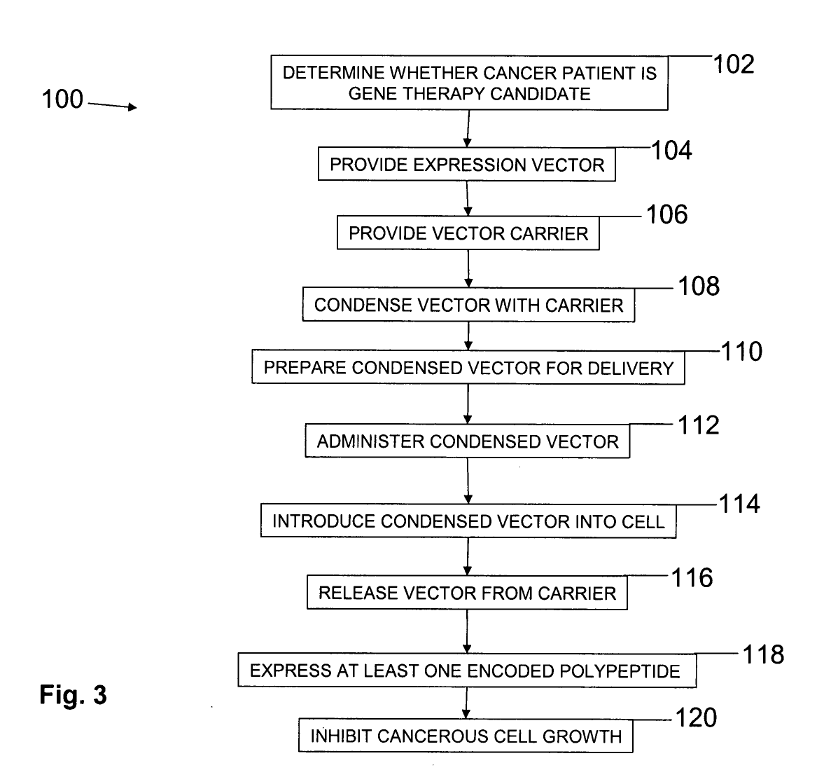

FIG. 3 is a flow diagram that illustrates an embodiment of a method 100 of inhibiting tumor growth in a patient, wherein the patient can be a human or any other animal gene therapy candidate. As such, a determination can be made as to whether or not the cancer patient is a candidate for gene therapy (Block 102). When the patient is a candidate for gene therapy, a combination expression vector can be provided that encodes for at least a therapeutic receptor and a therapeutic cytokine (Block 104). Additionally, a suitable vector carrier can be provided that is able to deliver an effective amount of gene into cells that can be induced to express the therapeutic polypeptides (Block 106). The carrier can be combined with the vector so as to condense the vector (Block 108), and the condensed vector can be prepared for delivery (Block 110). Preparing a condensed vector for delivery to a patient can include preparing the condensed vector into a composition that can be administered to the patient as is well known in art.

Additionally, after the vector is formulated for administration, it can be administered to the patent (Block 112). The vector can be administered to the patient by any route well known in the art, but injection into the patient is most preferred. As such, the composition having the condensed vector can be prepared for injection into any reasonable site in the patient, which can include injecting the composition by injection into a vein (intravenously), into a muscle (intramuscularly), into the space around the spinal cord (intrathecally), beneath the skin (subcutaneously), into a tumor (intratumorally), and the like. After the condensed vector has been administered, it can be introduced into a cell (Block 114). The condensed vector can enter the cell via endocytosis, receptor-mediated endocytosis, pinocytosis, and the like. While it can be advantageous for the condensed vector to enter a cancer cell, other types of cells that are proximate or remote from the tumor can also provide a therapeutic benefit; however, entry into cancerous cells is most preferred.

The combination expression vector can be released from the carrier at any point within the cell so that the encoded polypeptides can be expressed (Block 116). However, it is preferred that neither the condensed vector nor combination expression vector enter into the lysosome because of the harsh conditions, which can degrade the DNA. When inside the cell and released from the carrier, at least one polypeptide can be expressed by the cell, and preferably at least a combination of the receptor and cytokine can be expressed (Block 118).

The expression of the receptor and cytokine can then provide functional polypeptides that have physiological effects against cancer. Usually, the receptor and/or cytokine are transferred from inside the cell, often from within the cell nucleus, to outside the cell. The receptor and/or cytokine can then circulate locally and/or systemically to induce a physiological anticancer effect, or induce such an anticancer effect locally. The receptor can induce an anticancer effect by interacting with an angiogenic factor, and the cytokine can induce an anticancer effect by inducing an anticancer immunological effect related to cytokines. The combination of inducing an anti-angiogenic physiological effect and a cytokine-base immunological effect can then inhibit cancerous cell growth (Block 120). More preferably, the tumor regresses or is killed.

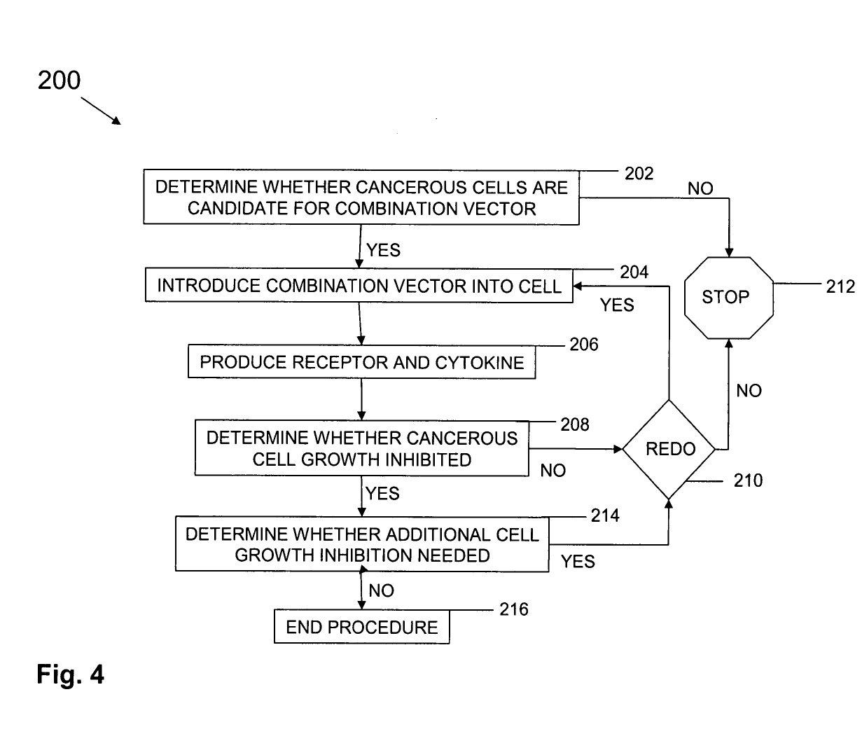

FIG. 4 is a flow diagram illustrating another embodiment of a method 200 of inhibiting cancerous cell growth. The method 200 can be implemented when a determination is made as to whether or not the cancerous cells are candidates for receiving a therapeutic combination expression vector (Block 202). In the instance the cancerous cells are candidates for gene therapy, a combination expression vector is introduced into a cell (Block 204), otherwise the method 300 is stopped (Block 212). The receptor and cytokine are then produced by the cell (Block 206), and it is determined whether or not the growth of the cancerous cells is inhibited (Block 208).

In the instance that the growth of cancerous cells is not inhibited, it is determine whether or not a redo should be performed (Block 210). A redo merely reintroduces the vector into a cell and the method 200 is repeated. A decision to not redo simply stops the method 200 (Block 212).

In the instance that the growth of cancerous cells is inhibited it is determined whether or not additional cancerous cell growth inhibition is needed (Block 214), when additional cancerous cell growth inhibition is needed, it is determined whether a redo is necessary (Block 210). On the other hand, when additional cancerous cell growth inhibition is not needed, the procedure can end (Block 216).

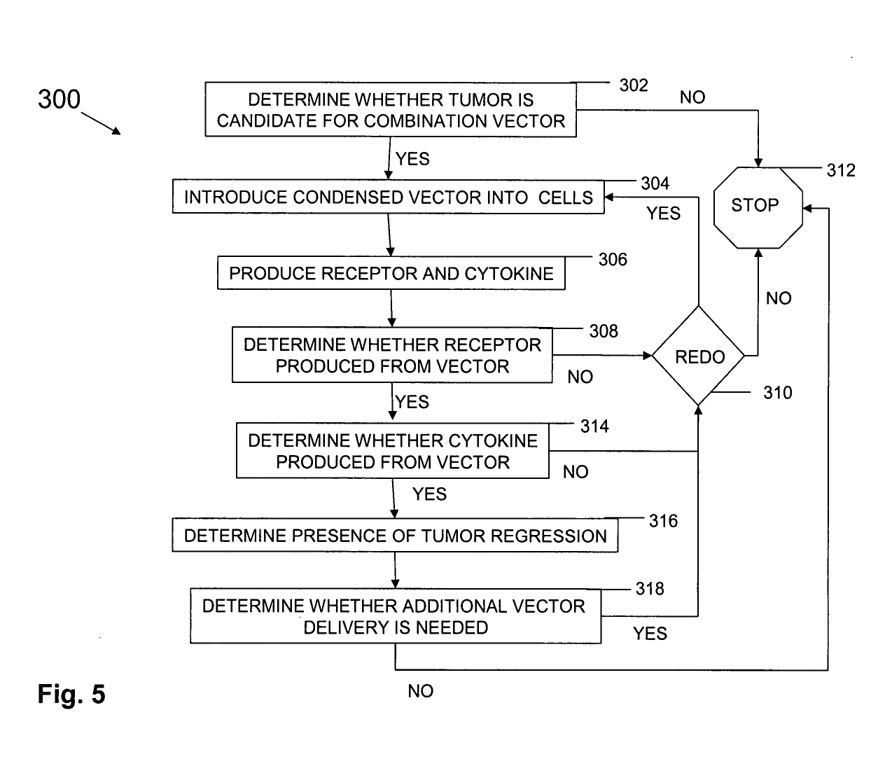

FIG. 5 is a flow diagram illustrating an embodiment of a method 300 for obtaining tumor regression with a combination expression vector. In the instance a tumor is determined to be a candidate for being treated with the combination expression vector the procedure is initiated (block 302), otherwise the method 300 is stopped (Block 312). The combination vector is then introduced into cells (Block 304), and the encoded receptor and cytokine are produced (Block 306).

Subsequently, it is determined whether or not the receptor has been produced or up-regulated (Block 308), and the option of a redo is assessed when the receptor has not been produced (Block 310). A redo merely reintroduces the vector into a cell and the method 300 is repeated. A decision to not redo simply stops the method 300 (Block 312). Also, it is determined whether or not the cytokine has been produced or up-regulated is needed (Block 314), and if not, another redo is assessed (Block 310).

Additionally, an assessment of tumor regression is performed (Block 316), and a determination of whether or not additional combination expression vector needs to be delivered is made (Block 318). In the instance that the tumor has regressed, but additional regression can be achieved, a redo may be initiated (Block 310). Also, when no tumor regression has been observed, the feasibility of a redo can be assessed (Block 310). On the other hand, when the tumor has completely regressed and has been effectively killed so that there is no longer any cancerous cell growth, the method can be stopped (Block 312).

The steps and/or acts of the foregoing methods can be interchanged and/or combined when feasible. As such, these methods only illustrate some of the embodiments of using the combination vectors in accordance with the present invention to treat cancer or inhibit cancer growth, especially in renal cell carcinoma. Accordingly, the use of a gene carrier for gene delivery can be used in any viable delivery technique. While cancerous cells in general can be targeted for growth inhibition by the expressed polypeptides, it is likely that the combination expression vectors can be delivered to the cancerous cells as well as other proximate and/or remote cells. In any event, any cell that can produce the polypeptides so as to have an anticancer effect on the cancerous cells can receive the combination expression vector. That is, the delivery of the combination expression vectors into cells other than the cancerous cells can elicit the therapeutic effect. In part, this is because the cytokines and angiogenic factor receptor can be locally and/or systemically circulated and produce a benefit local and/or remote with respect to the location of delivery and/or expression.

### EXAMPLES

The following examples illustrate embodiments of the invention, and are not intended to be limiting. As such, the following examples illustrate protocols that can be employed in order to practice the present invention. In the following examples, well-known biotechnological protocols were substantially followed as recommended by suppliers. The data collected from the following experiments were statistically analyzed using Prism 4.0 (Graphpad Software Inc, San Diego, Calif.). All statistical analyses were done using one-way ANOVA with Tukey's post-hoc test. Survival data was done using a Kaplan-Meier Survival analysis.

### Example 1

Expression vectors having a coding nucleotide sequence encoding for IL-2 were prepared by converting mRNA isolated from the Jurkat cell line and 16.5d mouse into cDNA using Oligo dT (Invitrogen, Burlingame, Calif.). Accordingly, PCR was performed under substantially standard conditions to amplify targeted polynucleotide sequences, wherein the targeted nucleotide sequences were obtained using the following primers: IL-2 Forward 5′-GTG CAG AAT TCA TCT ACA GGA TGC AAC-3′ (SEQ ID No: 9); and IL-2 Reverse 5′-CAC AAC GTC GAC TAA GTC AGT GTT GAG-3′ (SEQ ID No: 10). The PCR polynucleotide products were separated using gel electrophoresis under substantially standard conditions. Select bands of polynucleotides were then digested by EcoRI and SalI restriction endonucleases (Promega, Madison, Wis.), and resultant digested fragments were ligated into the pCI plasmid (Promega, Madison, Wis.) to obtain the desired pCMVIL-2 plasmid DNA expression vector. The pCMVIL-2 expression vector was confirmed by nucleotide sequencing.

### Example 2

Expression vectors having a coding nucleotide sequence encoding for sFlk-1 (soluble murine Flk-1) were prepared by converting mRNA isolated from the 16.5d mouse into cDNA using Oligo dT (Invitrogen, Burlingame, Calif.). Accordingly, PCR was performed under substantially standard conditions to amplify targeted polynucleotide sequences, wherein the targeted nucleotide sequences were obtained using the following primers: sFlk-1 Forward 5′-GAC GAA TTC ATG GAG AGC AAG GCG CTG CTA-3′ (SEQ ID No: 11); and sFlk-1 Reverse 5′-CTC TAG ACC ACC AAA GAT TTC ATC CCA C-3′ (SEQ ID No: 12). The PCR polynucleotide products were separated using gel electrophoresis under substantially standard conditions. Select bands of polynucleotides were then digested by EcoRI and SalI restriction endonucleases, and resultant digested fragments were ligated into the pCI plasmid (Promega, Madison, Wis.) to obtain the desired pCMVsFlk-l plasmid DNA expression vector. The pCMVsFlk- I expression vector was confirmed by nucleotide sequencing. Equipment used was the PTC-100 Thermocycler (MJ Research Inc., Watertown, Mass.). The primers were supplied by the University of Utah Core Laboratories, University of Utah. Sequencing was done by the University of Utah Core Laboratories, University of Utah.

### Example 3

The pCMVIL-2 and pCMVsFlk-1 plasmids as prepared in Examples 1 and 2, respectively, were used to prepare a combination expression vector encoding for IL-2 and sFlk-1. The Flk-1 cassette was excised from the pCMVsFlk-1 plasmid using BglII and BamHI restriction endonucleases. The pCMVIL-2 plasmid was opened by being digested with BglII restriction endonuclease. The msFlk-1 cassette was then inserted into the pCMVIL-2 plasmid at the BglII site by ligation to obtain a p2CMVFlk-1/IL-2 combination expression vector using T4 DNA Ligase (Promega, Madison, WI). The p2CMVFlk-1/IL-2 plasmid was confirmed by nucleotide sequencing, which is presented in the Sequence Listing as SEQ ID No: 1. Nucleotide sequencing was done by the University of Utah Core Laboratories, University of Utah.

### Example 4

The expression vectors pCMVIL-2, pCMVsFlk-1 and p2CMVFlk-1/IL-2 were prepared in accordance with Examples 1, 2, and 3, respectively and formulated for in vitro delivery. The expression vectors were complexed with PEI-g-PEG-RGD1.3 at 10:1 N/P. The formulations were configured to have an isotonic osmolality. The RGD peptide, ACDCRGDCFC, was purchased from the Genemed Synthesis, Inc. (San Franscisco, Calif.). After synthesis, peptides were purified via reverse phase high performance liquid chromatography (HPLC) and then analyzed by mass spectrometry, which was performed using matrix-assisted laser desorption/ionization time of flight (MALDI-TOF) mass spectrometer. The reagents, including branched PEI (average molecular weight 25kDa, average degree of polymerization 580), triethylamine (TEA), anhydrous N,N-dimethylformamide (DMF), and anhydrous diethyl ether, were purchased from Sigma- Adrich (Milwaukee, Wis.). N-hydroxysuccinimide-vinyl sulfone polyethylene glycol (NHS-PEG-VS; molecular weight 3400) was purchased from NEK-TAR (Huntsville, Ala.).

The synthesis of PEI-g-PEG-RGD conjugates consists of two reaction steps, as described. Briefly, in the first reaction step, RGD peptide was dissolved in anhydrous DMF containing 4 molar excess of TEA. NHS-PEG-VS was also dissolved in anhydrous DMF, and immediately mixed with 1 or 2 molar excess of peptide. After a 2-h incubation at room temperature, cold anhydrous ether was added into the reaction solution so that both RGD-PEG-VS conjugate and free peptide were precipitated out as a white power. After drying the precipitate under vacuum, it was dissolved in a pH 9.0 sodium carbonate buffer and filtered through the 0.22 Am syringe filter in order to remove un-conjugated free peptide. In the second reaction step, 1 or 2 molar excess of RGD-PEG-VS conjugates were mixed with the PEI solution in a pH 9.0 sodium carbonate buffer, depending on the desired conjugation degree, and incubated at room temperature overnight. The final product, PEI-g-PEG-RGD conjugate, was separated by dialysis and lyophilized. The composition including the PEI-g-PEG-RGD conjugates was monitored by ¹HNMR, at the reaction step of peptide with NHS-PEG-VS and at the conjugation step of PEI with RGD-PEG-VS. The NMR spectra were obtained on a Varian Inova 400 MHz NMR spectrometer (Varian, PaloAlto, Calif.) using standard proton parameters. Chemical shifts were referenced to the residual HDO resonance at approximately 4.7 ppm.

### Example 5

In vitro transfection efficiency for the delivery of the combination expression vector p2CMVFlk-1/IL-2 encoding for sFlk-1 and IL-2 was studied compared to an expression vector pCMVsFlk-1 encoding for sFlk-1, expression vector pCMVsFlt-1 encoding for sFlt-1, and an expression vector pCMVIL-2 encoding for IL-2. Accordingly, CADMEC cells were propagated and grown in RPMI-1640 growth media (Invitrogen) supplemented with 10% FBS and 1% pen/strep. The CADMEC cells were cultivated upon obtaining 70% confluency by using TrypLE (Invitrogen). The CADMEC cells were seeded (7.5×10⁴ cells) into 6-well plates and allowed to grow for about 24 hours in order to achieve about 60% confluency. After growing about 24 hours the growth media was replaced with RPMI-1640. The CADMEC cells were transfected by having about 2 μg of the naked plasmid or plasmid/polymer complexes, which were prepared in accordance with Example 4, added to the wells. Controls were maintained by well receiving no DNA or polymer and wells receiving only the polymer. After 4 hours, the media containing any excess plasmid/polymer complexes was removed and replaced with growth media. The supernatant was collected 48 hours later and IL-2 protein expression was measured by ELISA (OptEIA Human IL-2 ELISA set, Pharmingen, San Jose, Calif.) and read on a Bio-Rad 410 microplate reader (Bio-Rad, Hercules, Calif.). Also, in vitro cells used were CADMEC cells. RENCA cells were used in in vivo applications.

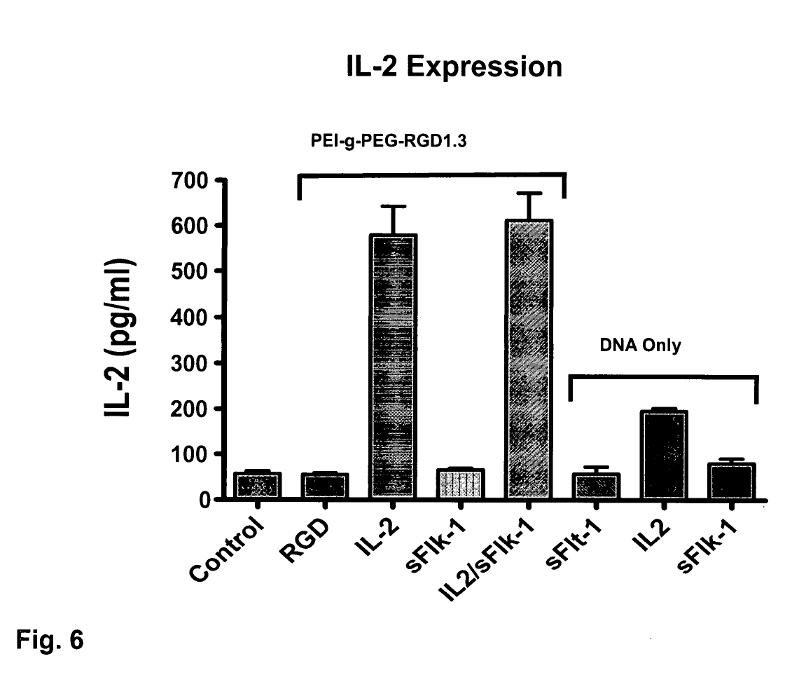

FIG. 6 is a bar graph illustrating the results of the foregoing in vitro transfections of CADMEC cells with pCMVIL-2, pCMVsFlk-1, pCMVsFlt-1, or p2CMVFlk-1/IL-2. The cells transfected with naked pCMVsFlk-1 or pCMVsFlt-1 did not show significant expression of IL-2 above the control values. Additionally, the cells transfected with the pCMVsFlk-1/polymer complex did not show significant expression of IL-2 above control values. The only significant elevation of IL-2 levels above controls was only demonstrated for pCMVIL-2 (DNA alone), pCMVIL-2, and p2CMVFlk-1/IL-2 carrier complexes. As shown, transfection with the polymer complexes exhibited about three-times the expression of naked DNA. The elevation of IL-2 levels above controls by transfection with pCMVIL-2 and p2CMVFlk-1/IL-2 carrier complexes was shown to be statistically similar by p<0.05 and p<0.0001, respectively. Thus, the combination expression vector p2CMVFlk-1/IL-2 induced elevated expression of IL-2 similar with the expression vector pCMVIL-2, thereby showing the combination expression vector to be therapeutically equivalent to single expression vector with respect to IL-2 production.

### Example 6

The expression vectors pCMVIL-2, pCMVsFlk-1 and p2CMVFlk-1/IL-2 were prepared in accordance with Examples 1, 2, and 3, respectively and formulated for in vivo delivery. The expression vectors were complexed with WSLP at 15:1 N/P. The formulations were configured to have an isotonic osmolality, and DNA at 0.5 μg/μL. Aliquots of the DNA/polymer complexes were prepared to have 25 μg of DNA in 50 μL of the formulation.

### Example 7

Tumor models to study in vivo transfection were prepared using RENCA cells delivered into BALB/c mice. Five-week old female BALB/c mice were purchased from Charles River and maintained on ad libitum rodent feed and water at room temperature and 40% humidity. All mice were acclimated to the environmental conditions for at least 1 week before tumor implantation. The BALB/c mice were injected with 1×10⁶ RENCA cells subcutaneously in the right flank, and the cells were allowed to grow and form a tumor model for 10 days. After 10 days, the tumors were measured and the mice sorted to allow for an average tumor size of 50 mm³-100 mm³ per group so that no group would have more than an 8 mm difference in tumor size. The tumor size in each mouse was measured every 3-4 days using a vernier caliper across its longest dimension (a) and shortest dimension (b). The approximate volume of each tumor was calculated using the formula V=0.5ab².

### Example 8

The in vivo transfection efficacy of the expression vector formulations including pCMVIL-2, pCMVsFlk-1, or p2CMVFlk-1/IL-2 complexed with WSLP as prepared in Example 6 were studied in RENCA tumor models. Accordingly, mice having the RENCA tumor model were prepared by subcutaneously injecting 1×10⁶ RENCA cells into the right flank of BALB/c mice and allowed to grow for 14 days, as in Example 7. The tumor bearing mice were injected locally with 50 μL of either 5% glucose or a DNA/WSLP complex having 25 pg of DNA. As such, the tumors received intratumoral injections of pCMVIL-2, pCMVsFlk-1, or p2CMVFlk-1/IL-2 complexed with WSLP every 7 days. Injections were terminated for all groups and the survival time was plotted. Mice were terminated when tumors became so large that they hindered movement or ulcerated.

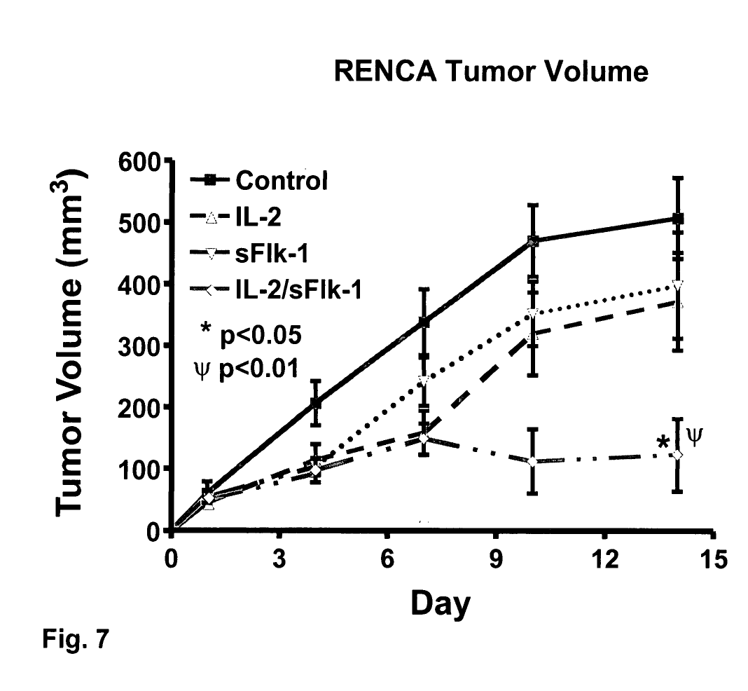

FIG. 7 is a graph illustrating the change in tumor volume when treated with 25 μg of pCMVIL-2, pCMVsFlk-1, or p2CMVFlk-1/IL-2 complexed with WSLP every 7 days. The control mice did not show any reduction in tumor volume, and the tumors had the largest increase in size throughout the duration of the experiment. The mice treated with pCMVIL-2 and pCMVsFlk-1 showed little decrease in tumor volume compared to controls after 14 days. Additionally, the mice treated with pCMVIL-2 and pCMVsFlk-1 showed similar tumor volumes throughout the duration of the experiment. The mice treated with p2CMVFlk-1/IL-2 showed tumor volume increases similar to the mice treated with pCMVIL-2 and pCMVsFlk-1 through 7 days after receiving only one injection. However, significant tumor growth inhibition was evident following the second injection of p2CMVFlk-1/IL-2 when compared to either glucose controls (p<0.01) or pCMVIL-2 or pCMVsFlk-1 (p<0.05). Additionally, four mice receiving the combination expression vector p2CMVFlk-1/IL-2 had no tumors two days following the second injection.

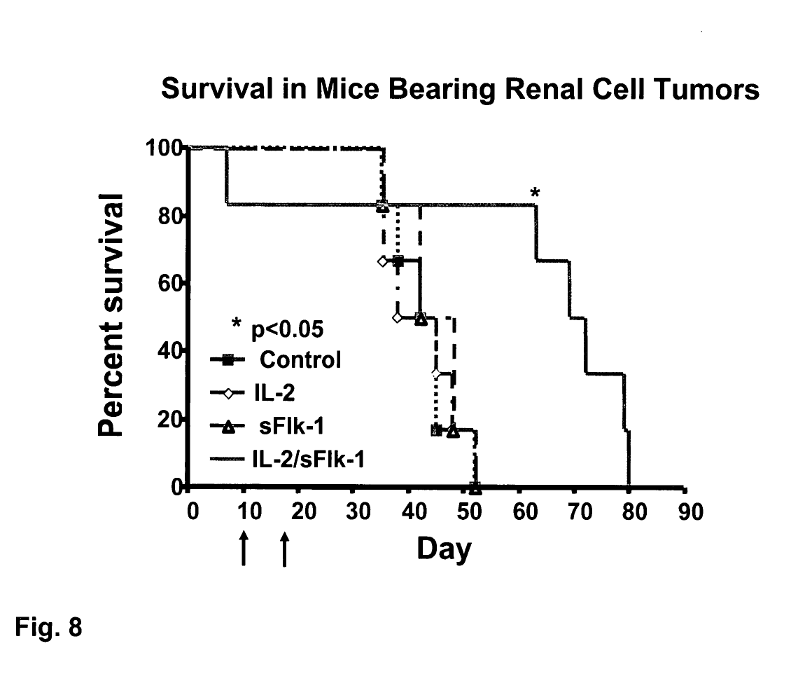

FIG. 8 is a graph illustrating the survival of mice bearing RENCA tumors after receiving the foregoing therapy. Accordingly, there was no difference in length of survival when given pCMVIL-2 or pCMVsFlk-1 when compared to controls. However, the mice receiving the combination expression vector p2CMVFlk-1/IL-2 demonstrated a 73% increase of median survival time, which extended up to 79 days. Ten days prior to the 1st treatment was for growth, two total injections were given marked by the two arrows at day 10 and 17.

### Example 9

The inhibition in growth and formation of lung metastases was studied in vivo. The expression vector formulations including pCMVIL-2, pCMVsFlk-1, or p2CMVFlk-1/IL-2 complexed with PEI-PEG-RGD1.3, as prepared in Example 4. The lung metastases model was prepared in BALB/c mice by having 1×10⁵ RENCA cells injected intravenously via the tail vein and allowed to grow for seven days. The test groups were given either 5% glucose solution (controls) or 40 μg of pCMVIL-2, pCMVsFlk-1, or p2CMVsFlk-1/IL-2 complexed with PEI-PEG-RGD1.3. The doses were administered at 200 μL volumes on day 8 post RENCA inoculation. The mice were weighed weekly to evaluate suppression or cessation of normal eating habits as well as monitored for signs of pain and distress according to IACUC recommendations.

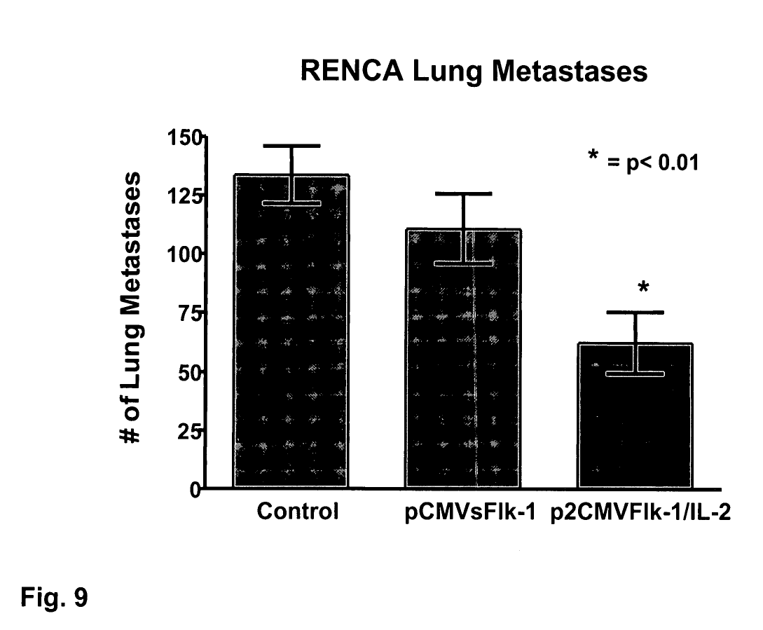

FIG. 9 is a bar graph illustrating the number of lung metastases formed during the foregoing experimental protocol. The pCMVIL-2/PEI-g-PEG-RGD1.3 complexes proved fatal two days following the initial injection, and are not graphically depicted. There was no statistically significant difference between the pCMVsFlk-1 plasmid treatment and the glucose control. However, there was a 54% reduction in tumor burden in animals treated with p2CMVsFlk-1/IL-2 over controls and 44% over pCMVsFlk-1 treated animals. Thus, the combination expression vector p2CMVsFlk-1/IL-2 inhibited lung metastases formation over any single expression vector.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An expression vector for use in inhibiting cancerous cell growth, the vector comprising:
a first promoter nucleotide sequence;
a first coding nucleotide sequence operatively linked to the first promoter sequence, the first coding nucleotide sequence encoding for a receptor that interacts with an angiogenic growth factor;
a second promoter nucleotide sequence upstream or downstream from the first promoter nucleotide sequence; and
a second coding nucleotide sequence operatively linked to the second promoter nucleotide sequence, the second coding nucleotide sequence encoding for a cytokine.

2. A vector as in claim 1, wherein the receptor inhibits angiogenesis by binding to the angiogenic growth factor.

3. A vector as in claim 2, wherein the receptor is selected from the group consisting of VEGF-R1, VEGF-R2, VEGF-R3, and derivatives thereof.

4. A vector as in claim 3, wherein the receptor is soluble.

5. A vector as in claim 4, wherein the first coding nucleotide sequence includes at least a portion thereof that hybridizes with a third coding nucleotide sequence consisting of SEQ ID NO: 4 or complement of SEQ ID NO: 4.

6. A vector as in claim 1, wherein the cytokine is selected from the group consisting of interleukins, chemokines, interferons, derivatives thereof.

7. A vector as in claim 6, wherein the second coding nucleotide sequence includes at least a portion thereof that hybridizes with a fourth coding nucleotide sequence consisting of SEQ ID NO: 6 or complement of SEQ ID NO: 6.

8. A vector as in claim 7, wherein at least one of the first promoter nucleotide sequence or second promoter nucleotide sequence includes at least a portion thereof that hybridizes with a third promoter nucleotide sequence consisting of SEQ ID NO: 8 or complement of SEQ ID NO: 8.

9. A vector as in claim 8, wherein the expression vector includes at least a portion thereof that hybridizes with a vector nucleotide sequence consisting of SEQ ID NO: 1 or complement of SEQ ID NO: 1.

10. An expression vector for use in inhibiting cancerous cell growth, the vector comprising:
a first promoter nucleotide sequence operatively linked to a first coding nucleotide sequence encoding for a first polypeptide that interacts with an angiogenic growth factor so as to inhibit angiogenesis;
a second promoter nucleotide sequence operatively linked to a second coding nucleotide sequence encoding for a second polypeptide that activates a physiological response against the cancerous cells, the second promoter sequence being upstream or downstream from the first promoter sequence, wherein the second coding nucleotide sequence is not operatively linked to the first promoter sequence and the first coding sequence is not operatively linked to the second promoter nucleotide sequence so that the first polypeptide is expressed independently of the second polypeptide sequence.

11. A vector as in claim 10, wherein the vector is characterized by the following:
the first polypeptide has a physiological activity substantially homologous with a first peptide sequence consisting of SEQ ID NO: 5; and
the second polypeptide has a physiological activity substantially homologous with a second peptide sequence consisting of SEQ ID NO: 7.

12. A vector as in claim 10, wherein the vector is characterized by the following:
the first coding nucleotide sequence includes at least a portion thereof that hybridizes with a third coding nucleotide sequence consisting of SEQ ID NO: 4 or complement of SEQ ID NO: 4; and
the second coding nucleotide sequence includes at least a portion thereof that hybridizes with a fourth coding nucleotide sequence consisting of SEQ ID NO: 6 or complement of SEQ ID NO: 6.

13. A vector as in claim 12, wherein at least one of the first promoter nucleotide sequence or the second promoter nucleotide sequence includes a portion thereof that hybridizes with a third promoter nucleotide sequence consisting of SEQ ID NO: 8 or complement of SEQ ID NO: 8.

14. A vector as in claim 13, wherein the vector further comprises at least one of the following:
a first chimeric interon between the first promoter nucleotide sequence and the first coding nucleotide sequence;
a second chimeric interon between the second promoter nucleotide sequence and the second coding nucleotide sequence;
a first T7-RNA polymerase promoter nucleotide sequence between the first promoter nucleotide sequence and the first coding nucleotide sequence;
a second T7-RNA polymerase promoter nucleotide sequence between the second promoter nucleotide sequence and the second coding nucleotide sequence;
a first poly-A tail nucleotide sequence downstream from the first nucleotide coding sequence;
a second poly-A tail nucleotide sequence downstream from the second nucleotide coding sequence;
a marker nucleotide sequence;
an antimicrobial nucleotide sequence; or at least one origin of replication nucleotide sequence.

15. A method of inhibiting growth of cancerous cells with an expression vector, the method comprising:
providing a combination expression vector including a first promoter nucleotide sequence operatively linked to a first nucleotide sequence encoding for a receptor that interacts with an angiogenic growth factor and a second promoter nucleotide sequence operatively linked to a second nucleotide sequence encoding for a cytokine, wherein the first promoter nucleotide sequence is upstream or downstream from the second promoter nucleotide sequence;
introducing the expression vector into at least one cell capable of expressing at least one of the receptor or cytokine; and
producing the receptor and cytokine.

16. A method as in claim 15, wherein the receptor is selected from the group consisting of VEGF-R1, VEGF-R2, VEGF-R3, and derivatives thereof and the cytokine is selected from the group consisting of interleukins, chemokines, interferons, derivatives thereof.

17. A method as in claim 16, wherein the receptor is soluble, and the receptor and cytokine are excreted extracellularly.

18. A method as in claim 16, wherein the receptor inhibits angiogenesis by binding with an angiogenic growth factor and the extracellular cytokine activates a physiological response against the cancerous cells, wherein the combination of the inhibition of angiogenesis and physiological response against the cancerous renal cells inhibits the growth of cancerous cells.

19. A method as in claim 18, wherein the receptor is VEGF-R2 or derivative thereof that inhibits angiogenesis by binding with an angiogenic growth factor, and the cytokine is IL-2 or derivative thereof that activates a physiological response against the cancerous cells.

20. A method as in claim 19, wherein the expression vector is characterized by the following:
the first coding nucleotide sequence includes at least a portion thereof that hybridizes with a third coding nucleotide sequence consisting of SEQ ID NO: 4 or complement of SEQ ID NO: 4;
the second coding nucleotide sequence includes at least a portion thereof that hybridizes with a fourth coding nucleotide sequence consisting of SEQ ID NO: 6 or complement of SEQ ID NO: 6; and
at least one of the first promoter nucleotide sequence or second promoter nucleotide sequence includes at least a portion thereof that hybridizes with a third promoter nucleotide sequence consisting of SEQ ID NO: 8.

21. A method as in claim 19, wherein the combination of VEGF-R2 or derivative thereof that inhibits angiogenesis and IL-2 or derivative thereof that induces the physiological response against the cancerous cells reduces a volume of a renal tumor comprised of the cancerous cells.


SEQUENCE LISTING (13 sequences)

SEQ ID NO: 1 - DNA, 6812 bp, Artificial
  The sequence is artificial by combining multiple genes of Flk-1 and IL-2.
  promoter (214)..(740): CMV Promoter
  Intron (857)..(989): Chimeric Interon
  promoter (1034)..(1052): T7-RNA Polymerase Promoter
  CDS (1069)..(2082): msFlk-1 Coding Sequence
  polyA_signal (2125)..(2345): Flk-1 PolyA Tail
  promoter (2576)..(3102): CMV Promoter
  Intron (3219)..(3351): Chimeric Interon
  promoter (3396)..(3413): T7-RNA Polymerase Promoter
  CDS (3431)..(3892): hIL-2 Coding Sequence
  polyA_signal (3917)..(4137): IL-2 PolyA Tail
  gene (4159)..(4227): LacZ Alpha Gene
  rep_origin (4233)..(4673): Filamentous Bacteriophage F1 Origin of Replication
  gene (5318)..(5977): AmpR Gene
  rep_origin (6075)..(6757): E. coli. ColE1 Origin of Replication
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcatggagagcaaggcgctgctagctgtcgctctgtggttctgcgtggagacccgagccgcctctgtgggtttgcctggcgattttctccatccccccaagctcagcacacagaaagacatactgacaattttggcaaatacaacccttcagattacttgcaggggacagcgggacctggactggctttggcccaatgctcagcgtgattctgaggaaagggtattggtgactgaatgcggcggtggtgacagtatcttctgcaaaacactcaccattcccagggtggttggaaatgatactggagcctacaagtgctcgtaccgggacgtcgacatagcctccactgtttatgtctatgttcgagattacagatcaccattcatcgcctctgtcagtgaccagcatggcatcgtgtacatcaccgagaacaagaacaaaactgtggtgatcccctgccgagggtcgatttcaaacctcaatgtgtctctttgcgctaggtatccagaaaagagatttgttccggatggaaacagaatttcctgggacagcgagataggctttactctccccagttacatgatcagctatgccggcatggtcttctgtgaggcaaagatcaatgatgaaacctatcagtctatcatgtacatagttgtggttgtaggatataggatttatgatgtgattctgagccccccgcatgaaattgagctatctgccggagaaaaacttgtcttaaattgtacagcgagaacagagctcaatgtggggcttgatttcacctggcactctccaccttcaaagtctcatcataagaagattgtaaaccgggatgtgaaaccctttcctgggactgtggcgaagatgtttttgagcaccttgacaatagaaagtgtgaccaagagtgaccaaggggaatacacctgtgtagcgtccagtggacggatgatcaagagaaatagaacatttgtccgagttcacacaaagccttttattgctttggtagtgggatgaaatctttggtggtctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcatgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 2 - protein, 337 aa, Artificial
  Synthetic Construct
MESKALLAVALWFCVETRAASVGLPGDFLHPPKLSTQKDILTILANTTLQITCRGQRDLDWLWPNAQRDSEERVLVTECGGGDSIFCKTLTIPRVVGNDTGAYKCSYRDVDIASTVYVYVRDYRSPFIASVSDQHGIVYITENKNKTVVIPCRGSISNLNVSLCARYPEKRFVPDGNRISWDSEIGFTLPSYMISYAGMVFCEAKINDETYQSIMYIVVVVGYRIYDVILSPPHEIELSAGEKLVLNCTARTELNVGLDFTWHSPPSKSHHKKIVNRDVKPFPGTVAKMFLSTLTIESVTKSDQGEYTCVASSGRMIKRNRTFVRVHTKPFIALVVG

SEQ ID NO: 3 - protein, 153 aa, Artificial
  Synthetic Construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - DNA, 1014 bp, Mus musculus
  CDS (1)..(1014): Flk-1 Coding Sequence
atggagagcaaggcgctgctagctgtcgctctgtggttctgcgtggagacccgagccgcctctgtgggtttgcctggcgattttctccatccccccaagctcagcacacagaaagacatactgacaattttggcaaatacaacccttcagattacttgcaggggacagcgggacctggactggctttggcccaatgctcagcgtgattctgaggaaagggtattggtgactgaatgcggcggtggtgacagtatcttctgcaaaacactcaccattcccagggtggttggaaatgatactggagcctacaagtgctcgtaccgggacgtcgacatagcctccactgtttatgtctatgttcgagattacagatcaccattcatcgcctctgtcagtgaccagcatggcatcgtgtacatcaccgagaacaagaacaaaactgtggtgatcccctgccgagggtcgatttcaaacctcaatgtgtctctttgcgctaggtatccagaaaagagatttgttccggatggaaacagaatttcctgggacagcgagataggctttactctccccagttacatgatcagctatgccggcatggtcttctgtgaggcaaagatcaatgatgaaacctatcagtctatcatgtacatagttgtggttgtaggatataggatttatgatgtgattctgagccccccgcatgaaattgagctatctgccggagaaaaacttgtcttaaattgtacagcgagaacagagctcaatgtggggcttgatttcacctggcactctccaccttcaaagtctcatcataagaagattgtaaaccgggatgtgaaaccctttcctgggactgtggcgaagatgtttttgagcaccttgacaatagaaagtgtgaccaagagtgaccaaggggaatacacctgtgtagcgtccagtggacggatgatcaagagaaatagaacatttgtccgagttcacacaaagccttttattgctttggtagtgggatga

SEQ ID NO: 5 - protein, 337 aa, Mus musculus; identical to SEQ ID NO: 2

SEQ ID NO: 6 - DNA, 462 bp, Homo sapiens
  CDS (1)..(462): IL-2 Coding Sequence
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 7 - protein, 153 aa, Homo sapiens; identical to SEQ ID NO: 3

SEQ ID NO: 8 - DNA, 527 bp, Cytomegalovirus Human Herpesvirus
  promoter (1)..(527): CMV Promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcaga

SEQ ID NO: 9 - DNA, 27 bp, Homo sapiens
  misc_feature (1)..(27): IL-2 gene forward primer
gtgcagaattcatctacaggatgcaac

SEQ ID NO: 10 - DNA, 27 bp, Homo sapiens
  misc_feature (1)..(27): IL-2 gene reverse primer
cacaacgtcgactaagtcagtgttgag

SEQ ID NO: 11 - DNA, 30 bp, Mus musculus
  misc_feature (1)..(30): Soluble murine Flk-1 gene forward primer
gacgaattcatggagagcaaggcgctgcta

SEQ ID NO: 12 - DNA, 28 bp, Mus musculus
  misc_feature (1)..(28): Soluble murine Flk-1 gene reverse primer
ctctagaccaccaaagatttcatcccac

SEQ ID NO: 13 - protein, 10 aa, Artificial
  This artificial peptide is an integrin-binding RGD peptide.
ACDCRGDCFC